(19) United States
(12) Patent Application Publication
(10) Pub. No.: US 2021/0387071 A1
(43) Pub. Date: Dec. 16, 2021

(54) Method and Apparatus for Leveling Metrics between Contestants in a Fitness Betting Challenge

(71) Applicants: James M. Miller, Bend, OR (US); Glennon P. Middleton, Bothell, WA (US); Shawn D. Beach, Bend, OR (US)

(72) Inventors: James M. Miller, Bend, OR (US); Glennon P. Middleton, Bothell, WA (US); Shawn D. Beach, Bend, OR (US)

(21) Appl. No.: 17/347,497

(22) Filed: Jun. 14, 2021

Related U.S. Application Data
(60) Provisional application No. 63/038,398, filed on Jun. 12, 2020.

(51) Int. Cl. A63B 71/06; A63B 24/00

(52) U.S. Cl. CPC A63B 71/0605; A63B 71/0686; A63B 24/0084; A63B 24/0062; A63B 2024/0065; A63B 2024/0068; A63B 2230/705; A63B 2220/20; A63B 2225/20; A63B 2225/50; A63B 2230/015; A63B 2230/062; A63B 2230/505; A63B 2220/12

(57) ABSTRACT

Tracking information is provided from one or more mobile communications devices indicating fitness activity recorded on personal fitness devices of multiple users over a predetermined baseline period of time. A mobile communications device of a challenging user communicates a fitness challenge that is to occur over a predetermined challenge period of time to one or more challenged users. Leveled challenged fitness metrics of the challenging user and of the challenged user is computed based on fitness activity of the challenging user and the challenged user that occurs during the challenge period of time and during the baseline period of time. The leveled challenged fitness metrics are communicated to the mobile communication devices upon completion of the challenge period of time to indicate a winner of the challenge.

This application claims the benefit of U.S. provisional application No. 63/038,398 filed on Jun. 12, 2020, the contents of which is hereby incorporated by reference.

### BACKGROUND

This application relates to systems and methods for leveling fitness metrics between contestants in a fitness betting challenge tied to desired activities centered on a fitness tracking system.

Fitness contests are available in application format are available on a tracking device in which one player accomplishes a challenge, and the result of such competition is shown to other players. Such challenges are on a level playing field where one competitor results are displayed and compared against another player performing the same fitness activity (e.g., performing a number of steps in a day). These fitness activities do not account for historical advantages that one competitor has over the other competitor, such as one competitor being a full-time athlete, and the other competitor competing part time. Thus the incentive for one or both competitors to increase their fitness activity is limited as the one competitor will unlikely be able to compete with the other competitor thereby reducing the incentive for the competitors to compete at a higher level.

### SUMMARY OF THE INVENTION

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions. One general aspect includes a method for metric leveling of personal fitness activity during a personal fitness wager event. The method also includes tracking with one or more personal fitness devices the fitness activity of multiple users. The method also includes communicating after a fitness activity from the one or more personal fitness devices to one or more mobile communication devices results of the tracking the fitness activity. The method also includes indicating with the one or more mobile communication devices end times of periodic time periods in which the fitness activity is to occur. The method also includes in response to the indication by the timer, communicating with the one or more communications devices via a communications network to one or more servers, at the end times of the periodic time periods aggregated results of the fitness activity over a periodic time period. The method also includes aggregating with one or more servers the results of the fitness activities occurring over a second predetermined time period and computing a baseline fitness activity per periodic time period for each of the multiple users. The method also includes entering by one of the multiple users a fitness challenge on one of the mobile communication devices, the fitness challenge indicating fitness goal for completion of at least one fitness activity over a third predetermined time period. The method also includes communicating to the one or more servers and the mobile communication devices via the communication network a challenge indication by one of the mobile communications devices indicating a fitness challenge by a challenging member of one of the multiple users. The method further includes communicating a challenge acceptance indication to the one or more servers via the communication network by one of the mobile communication devices acceptance of the fitness challenge by a challenged user of one of the multiple users. The method also includes communicating by the server via the communication network to the mobile communications device of the challenged user a leveled fitness metric to a challenged user, the leveled fitness metric determined based on the baseline fitness activity per periodic time period for the challenging user and the baseline fitness activity per periodic time period for the challenged user. The method also includes storing in the server an indication of the duration of the challenge and setting with the server a timer to indicate the end of the challenge based on the stored duration. The method also includes in response to setting the timer, communicating by the server via the communication network to the mobile communications device of the challenged user and the challenged user an indication of initiation of the challenge and the challenge duration. The method also includes tracking with one or more personal fitness devices the fitness activity of the challenging user and the challenged user. The method also includes communicating after a fitness activity from the one or more personal fitness devices to one or more mobile communication devices of the challenging user and the challenged user results of the tracking the fitness activity. The method also includes communicating with the one or more communications devices of the challenging user and the challenged user via a communications network to one or more servers, the fitness activity occurring during the duration of the challenge of the challenging user and the challenged user. The method also includes in response to the timer indicating the end of the challenge, aggregating with one or more servers the results of the fitness activities of the challenge of the challenging user and the challenged user occurring during the duration of the challenge and computing a challenged fitness metric of the challenging user and of the challenged user based on fitness activity of the challenging user and the challenged user during the duration of the challenge and based on the baseline fitness activity per periodic time period for the challenging user and of the challenged user. The method also includes indicating a winner to the mobile communication device of the challenging user and of the challenged user based on the challenged fitness metric.

In an additional embodiment an apparatus is provided that includes a computer system having a timer circuit providing a signal indicating a predetermined baseline period of time, and computer-readable media storing program code. The computer readable media when executed on the computer system performs the functions of determining from the signal the predetermined baseline period of time, receiving a tracking signal from mobile communications devices indicating fitness activity recorded on personal fitness devices of multiple users over the predetermined baseline period of time, communicating a signal containing a challenge indication by the mobile communications devices indicating a fitness challenge to occur over a predetermined challenge period of time by a challenging user of one of the multiple users to a challenged user of one of the multiple users, computing challenged fitness metrics of the challenging user and of the challenged user based on fitness activity of the challenging user and the challenged user occurring during the challenge period of time and during the baseline period of time, and communicating to the mobile communication devices a signal indicating the challenged fitness metrics upon completion of the challenge period of time to indicate a winner of the challenge.

In another implementation a system for leveling metrics between contestants in a fitness betting challenge includes personal fitness devices to be worn by a plurality of users. The fitness devices include a device for determining with a timer circuit a predetermined baseline period of time, recording fitness activity of one of the plurality of users with one of a plurality of fitness devices during the predetermined baseline period of time and transmitting a signal indicating the recorded fitness activity. The mobile communications devices have a receiver to receive from one of the fitness devices a signal indicating fitness activity recorded on the one of the personal fitness devices during the predetermined baseline period of time and has a transmitter to transmit a signal with a challenge indication indicating a fitness challenge during a predetermined challenge period of time by a challenging user to a challenged user. A server computer is provided that includes a receiver to receive a tracking signal from one of the mobile communications devices indicating fitness activity recorded on one of the personal fitness devices, a transmitter to communicate the signal with the challenge indication by one of the mobile communications devices indicating a fitness challenge during the predetermined challenge period of time by a challenging user to a challenged user. A processor is included to compute challenged fitness metrics of the challenging user and of the challenged user based on fitness activity of the challenging user and the challenged user occurring during the challenge period of time and during the baseline period of time, A transmitter is provided to communicate to one of the mobile communication devices a signal indicating the challenged fitness metrics upon completion of the challenge period of time and an indication of a winner of the challenge. Other embodiments of this aspect include corresponding computer systems, apparatus, and computer programs recorded on one or more computer storage devices, each configured to perform the actions of the methods.

### BRIEF DESCRIPTION OF THE DRAWINGS

FIG. 1 is a simplified schematic diagram of a system for leveling fitness metrics between contestants in a fitness betting challenge;

FIG. 2 is a simplified schematic diagram of an exemplary computing device used in the system for leveling metric between contestants in a fitness betting challenge;

FIG. 3 is a simplified schematic diagram of an exemplary hosting computing device used in the system for leveling metrics between contestants in a fitness betting challenge; and

FIG. 4 illustrates a flow diagram of a process for leveling Metrics between contestants in a fitness betting challenge.

### DETAILED DESCRIPTION

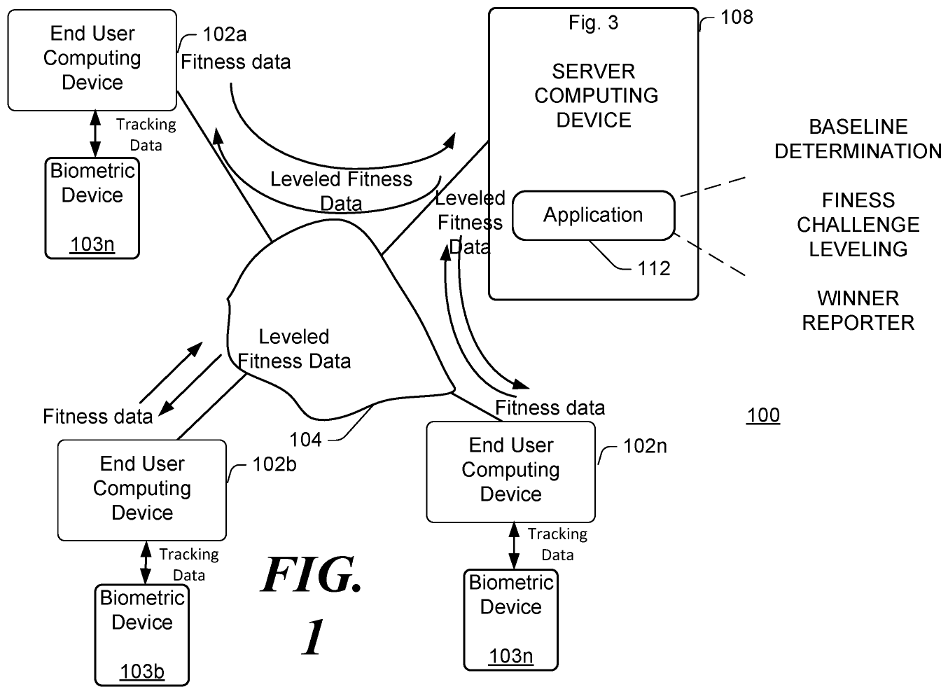

Referring to FIG. 1 there is shown a system for leveling metrics between contestants in a fitness betting challenge 100 including end user computing devices (also referred to herein as “personal computing devices” or “mobile communication devices”) 102a-102n coupled via a network 104 to one or more network server devices 108. In one implementation, each of the End User computing devices 102a-n is connected to a respective one of Biometric Device 103 (a-n). Biometric devices 103 (a-n) may be attached to a user or periodically attached to a user to monitor biometric/medical/wellness and/or fitness activities of the user. Exemplary biometric devices 103 (a-n) include a smart watch, a fitness tracking device, a bio-feedback device, or a biometric tracking device. The biometric devices (also referred to herein as “personal fitness devices”) 103a-n may include a display and may include a global positioning sensor GPS to detect distance traveled by one of the multiple users (and may include other sensors as described herein) during a fitness activity. Such activity may be communicated with steps of the user.

Server computing device 108 is described communicating directly with computing devices 102a-102n; however, such communication is for illustration purposes only and in a typical implementation server computing device 108 may communicate via network 104 to end user computing devices 102(a-n), other end user computing devices (not shown), and/or directly to a biometric devices 103a-n.

Server computing device 108 may be a network computer, host computer, network server, web server, email server or any computing device for hosting email communications applications and systems, one example of which includes a Microsoft® exchange server. Although end user computing devices 102(a-n) and other client computing device are described as a personal computing device, devices 102(a-n) may be any type of computing device such as a cell phone, smart phone, smart watch, laptop, mobile computer, desktop computer, personal computer, PDA, music player or game player device.

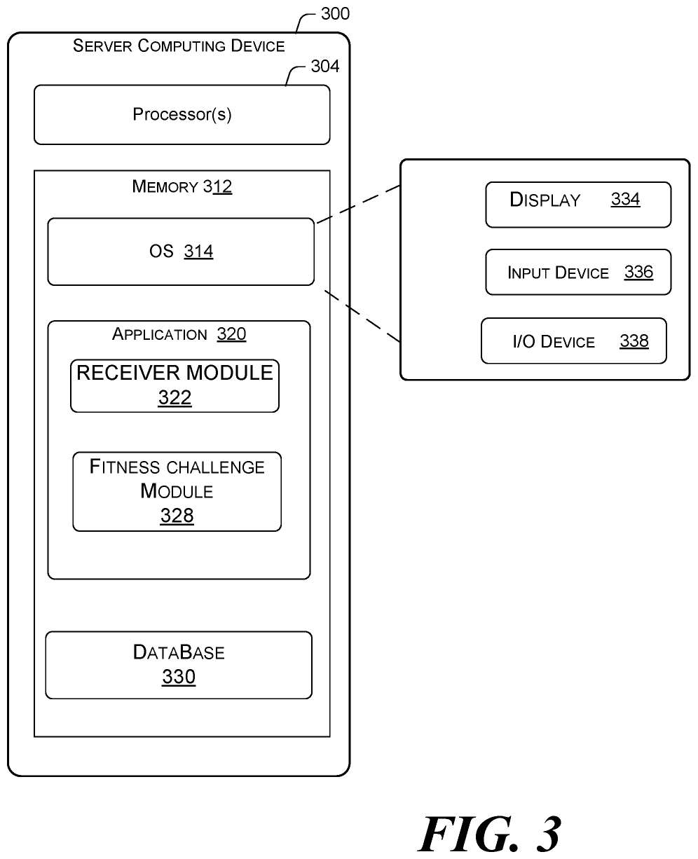

In one implementation, server computing device 108 includes one or more processors (See FIG. 3) and computer memory containing software application 112 which when executed by the processors, allows leveling fitness metrics between contestants in a fitness betting challenge between the end user computing devices 102(a-n) via the network, to receive indications from biometric equipment 103(a-n) used by the end users, and display the results. The indications from the end user computing devices 102(a-n) may be an indication of a biometric tracking of a fitness event over a pre-determined period of time.

The server computing device 108 may receive a tracking from one or more of the mobile communications devices 102a-n indicating fitness activity recorded on personal fitness devices 103a-n of multiple users over a predetermined baseline period of time (e.g., 30-day period). One of the mobile communications devices may communicate a challenge indication by indicating a fitness challenge (e.g., complete 10 k steps per day) over a predetermined challenge period of time (e.g., 10-day period) by a challenging user (e.g., user of end user computing device 102a) of one of the multiple users to a challenged user (e.g., user of computing device 102b) of one of the multiple users. The personal fitness device 103a-n may include a watch or other computing device with a display and may include a global positioning sensor to detect distance traveled by one of the multiple users during a fitness activity. Such activity may be communicated with steps of the user.

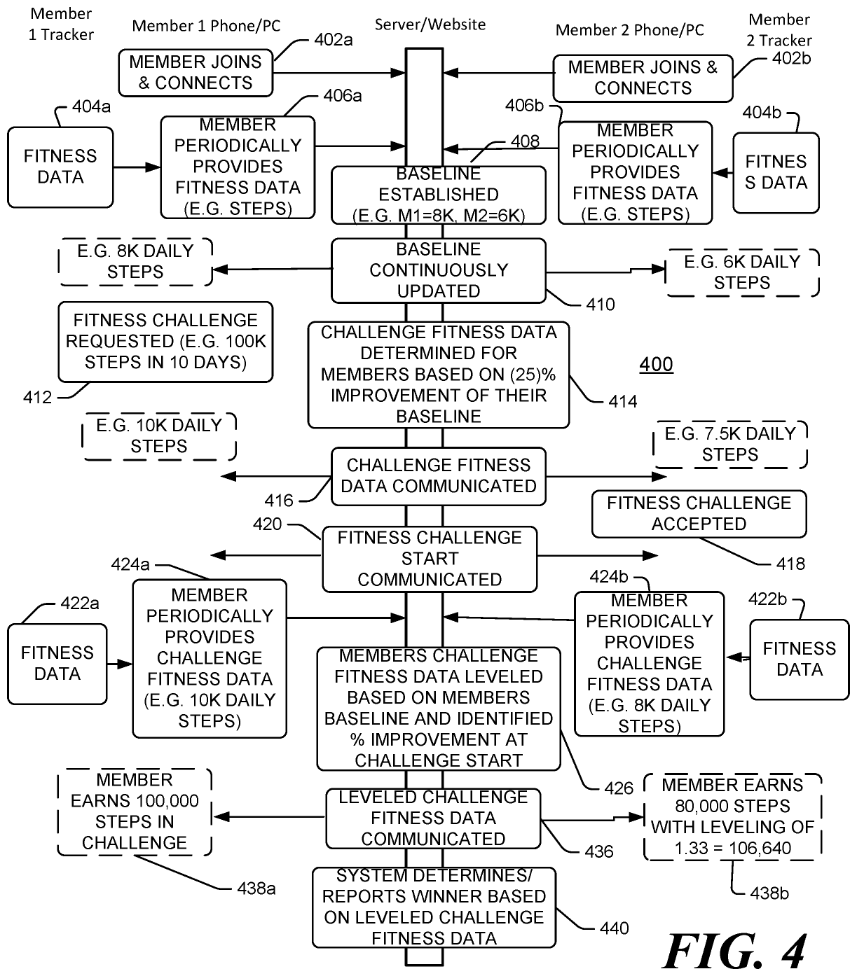

The server computing device 108 may compute and level challenged fitness metrics of the challenging user and of the challenged user based on fitness activity of the challenging user and the challenged user occurring during the challenge period of time (10-day challenge period) and during the baseline period of time (30-day period). Further details of this computation and leveling is described in connection with FIG. 4. The server computing device 108 may communicate to the one or more mobile communication devices 102a-n one or more of the level challenged fitness metrics upon completion of the challenge period of time to indicate a winner of the challenge.

Although only three end user computer devices 102a-102n, are described, the server computing device 108 may send notifications of entries in the sweepstakes to the end user computing devices, or to any device on the internet, a company intranet, or the World Wide Web.

The server computing device 108 receives indications via network 104 from end user computing device 102a and end user computing device 102(b-n). In one implementation when device 102a indicates a desired completion, it sends a unique identification code identifying that the request came from computing device 102a. Also when device 102b provides an indication, it sends a unique identification code identifying that the request came from computing device 102b. Server 108 and Computing devices 102a-n have internal timers (such as time logic and a crystal clock) that track time periods and provides circuitry to provide an indication after expiration of the time periods.

In another implementation computing device 108 responds to the request from devices 102a by providing an indication of the start of the challenge via network 104 to device 102a-n, and responds to indications of the result (metrics) of the fitness challenge from devices 102a-n by leveling the metrics of the challenge, and feeding the leveled metrics via network 104 to the devices 102a-n.

Server computing 108 tracks the metrics. In addition, server computing device 108 is programmed with the criteria required to compute a winner of the fitness challenge.

### Example Personal Computing Device Architecture

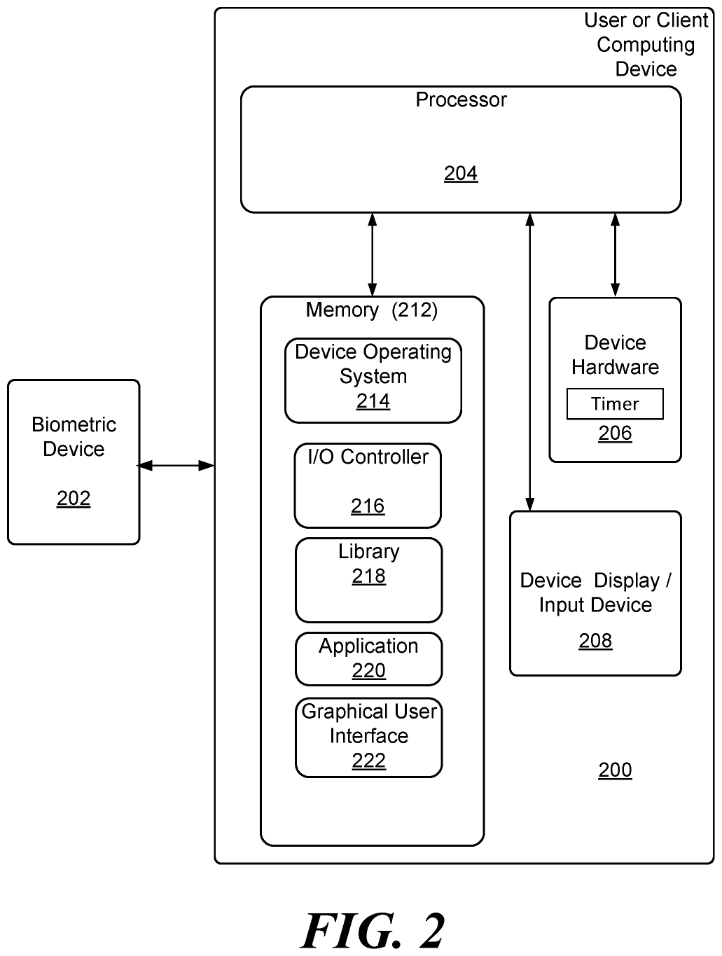

In FIG. 2 there are illustrated selected modules in Personal Computing Device 200 (computing devices 102a-n of FIG. 1) and biometric device 202 (biometric devices 103a-n of FIG. 1). Personal computing device 200 includes a processing device 204, memory 212, hardware 206 and display/input device 208. Hardware 206 includes an internal clock/timer 207 (e.g., a crystal connected to count up timer logic, also referred to herein as a “timer circuit”) to determine and indicate predetermine time periods. Processing device or processor 204 may include a microprocessor, microcontroller or any such device for accessing memory 212, device hardware 206 and display/input device 208. Processing device 204 has processing capabilities and memory suitable to store and execute computer-executable instructions. In one example, processor 204 includes one or more processors.

Biometric device 202 connects to computing device 102 directly via a wire or wirelessly. Biometric device 202 monitors activities/wellness of a user to which it is attached. Examples of biometric device 103 include a heart rate monitor, a pedometer (to track distance travelled and steps taken), a GPS (global positing system) tracking device, a stopwatch, a blood level monitoring device (for measuring LDH, insulin levels or other blood levels), blood pressure monitor, body temperature monitor, caloric intake monitor, body fat percentage monitor, or a weight monitor. The biometric device 202 may be programmed to provide an indication on a particular event—such as when a blood level reaches a predetermined parameter for a predetermined period of time, when a user's heart rate is within a particular zone for a predetermined period of time more than a predetermined number of times a week, when a step goal has been achieved or when steps have been accumulated for a period of time, when active minutes have been reported or activity goals detected and achieved.

Processing device 204 executes instructions stored in memory 212, and in response thereto, processes signals from hardware 206 and display/input device 208. Hardware 206 may include network and communication circuitry for communicating with network 104 (FIG. 1) and a time/clock for indicating the expiration of a predetermined period of time. Display/Input device 208 receives inputs from a user of personal computing device 102a-n (See FIG. 1) and may include a keyboard, mouse, track pad, microphone, audio input device, video input device, or touch screen display. Display device 208 may include an LED (Light emitting diode), LCD (Liquid crystal display), CRT (cathode ray tube) or any type of display device.

Memory 212 may include volatile and nonvolatile memory, removable and non-removable media implemented in any method or technology for storage of information, such as computer-readable instructions, data structures, program modules or other data. Such memory includes, but is not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, RAID storage systems, or any other medium (including a non-transitory computer readable storage medium) which can be used to store the desired information, and which can be accessed by a computer system.

Modules stored in memory 212 of the personal computing device 200 may include an operating system 214, an I/O controller 216, a library 218, a browser application 220 and a graphical user interface 222. Operating system 214 may be used by application 220 to operate device 200. I/O controller may provide drivers for device 200 to communicate with hardware 206 or device 208. Library 218 may include preconfigured parameters (or set by the user before or after initial operation) such as personal computing device operating parameters and configurations. Browser application may include a generally known network browser (including, but not limited to, Internet Explorer, Netscape Firefox, Oracle, Chrome, or Safari) for displaying articles manifested as web pages received from the network or indications from Biometric device 202.

### Example Architecture

In FIG. 3 there are illustrated selected modules in host computing device 300 (Server Computing Device 108 of FIG. 1) using process 400 shown in FIG. 4. Hosting device 300 includes a processing device 304, memory 312, and hardware 314. Processing device 304 may include one or more microprocessors, microcontrollers or any such devices for accessing memory 312 or hardware 314. Processing device 304 has processing capabilities and memory 312 suitable to store and execute computer-executable instructions.

Processing device 304 executes instruction stored in memory 312, and in response thereto, processes signals (e.g., binary logic levels) from hardware 314. Hardware 314 may include a display 334, and input device 336 and an I/O device 338. I/O device 338 may include a network and communication circuitry for communicating with network 104. Input device 336 receives inputs from a user of the host computing device 300 and may include a keyboard, mouse, track pad, microphone, audio input device, video input device, or touch screen display. Display device 334 may include an LED, LCD, CRT, or any type of display device.

Memory 312 may include volatile and nonvolatile memory, removable and non-removable media implemented in any method or technology for storage of information, such as computer-readable instructions, data structures, program modules or other data. Such memory includes, but is not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, RAID storage systems, or any other medium which can be used to store the desired information, and which can be accessed by a computer system.

Stored in memory 312 of the hosting device 300 may include an operating system 314, application 320 and a library of other applications such as a database 330. Operating system 314 may be used by application 320 to operate device 300. The operating system 314 may include drivers for device 300 to communicate with input device 336 and I/O device 338. Database 330 may include preconfigured parameters (or set by the user before or after initial operation) such web site operating parameters, web site libraries, HTML (Hyper Text Markup Language) libraries, API's (Application program interface) and configurations.

Stored in the database in memory 312 are the parameters regarding the sweepstakes, the type of biometric devices supported, drivers for the biometric devices and information regarding the users—subscribers.

Application 320 includes a receiver module 322 and a fitness challenge module 328. Receiver module 324 and fitness challenge module 328 are described in FIGS. 4 in process 400 for metric leveling between contestants in a fitness challenge. The exemplary process in FIG. 4 is illustrated as a collection of blocks in a logical flow diagram, which represents a sequence of operations that can be implemented in hardware, software, and a combination thereof. In the context of software, the blocks represent computer-executable instructions that, when executed by one or more processors, perform the recited operations. Generally, computer-executable instructions include routines, programs, objects, components, data structures, and the like that perform particular functions or implement particular abstract data types. The order in which the operations are described is not intended to be construed as a limitation, and any number of the described blocks can be combined in any order and/or in parallel to implement the process. For discussion purposes, the processes are described with reference to FIG. 4, although it may be implemented in other system architectures.

Referring to FIG. 4, a flowchart of process 400 performed by processor 304 when executing the software instructions in application 320 is shown. Process 400 includes blocks 402-422. When application 320 is executed on the host processing device 300, it uses the processor 304 and instructions in modules 322-338 that are shown in FIG. 3.

In the process 400, the server computing device 300 (FIG. 3) in block 402 sets parameters for leveling of fitness metrics between contestants (referred to herein as “users” or “members”) is set to be tied to performance of physical activities in a fitness betting challenge or wager event.

In block 402a, a member (e.g., member 1) using a member personal computing device (such a smart phone or a personal computer) connects via the web to the server, and logs into the fitness challenge website.

In block 402b, a member (e.g., member 2) using a member personal computing device (such a smart phone or a personal computer) connects via the web to the server, and logs into the fitness challenge website.

In blocks 404a and 404b, a fitness device tracks biodata or other fitness activity (e.g., steps) about one of the members to which it is connected (e.g., member 1 and member 2 respectively), and provides signals containing the fitness data, including results of tracked fitness activity to the member's respective member personal computing device.

In block 406a and 406b, each of the member personal computing devices (mobile communication device) respectively provides a signal indicating the fitness data to the server via a telecommunication network.

In block 406a and 406b, the one or more mobile communication devices determine using a timer (e.g., timer 207), end times of periodic time periods in which the fitness activity is to occur. In response to the indication by the timer, the one or more communications devices communicate via the communications network to one or more servers, at the end times of the periodic time periods a signal indicating aggregated results of the fitness activity over a periodic time period.

In box 408, the server establishes a baseline by aggregating with the one or more servers the results of the fitness activities occurring over a first predetermined time period and computing a baseline fitness activity per periodic time period for each of the multiple users (e.g., 8 k daily steps for member 1, and 6 k daily steps for member 2).

In box 410, server in response to receiving new daily updates from the mobile communications devices, updates the established baseline.

In box 412, one of the multiple users enters a fitness challenge on one of the mobile communication devices (e.g., mobile device 1), the fitness challenge indicates a personal fitness goal for completion of at least one fitness activity over another predetermined time period (e.g., 10 k daily steps over 10 days).

In box 414, the server determines a challenge fitness data improvement for all the members based on the improvement (member 1 fitness challenge request divided by member 1 baseline) of the member requesting the challenge (e.g., member 1). The improvement is the requested challenge improvement of the challenging member (e.g., member 1 divided by the baseline data of member 1). For member 1 requesting 10 k steps would be a 25% improvement. For member 2 and all other members the daily fitness activity to meet the challenge based on each member baseline data is determined. For example, a 25% improvement for member 2 is 7.5 k daily steps (6 k×1.25).

In block 416, the one or more servers communicates to the mobile communication devices via the communication network a signal with the challenge indication by one of the mobile communications devices indicating a fitness challenge by a challenging member of one of the multiple users.

In block 418, a signal with a challenge acceptance indication is communicated to the one or more servers via the communication network by one of the mobile communication devices acceptance of the fitness challenge by a challenged user of one of the multiple users.

In block 420, a start fitness challenge is communicated to the members participating in the challenge (e.g., member 1 and member 2). The server stores an indication of the duration of the challenge and setting with the server a timer (e.g., timer 207) to indicate the end of the challenge based on the stored duration. In response to setting the timer, the server communicates via the communication network to the mobile communications device of the challenged user and the challenged user a signal with an indication of initiation of the challenge and the challenge duration.

In block 422a member 1 tracker during a challenge time interval provides fitness data (e.g., 10 k steps per day) to the member 1 (the challenging member) mobile communication device, and in block 422b member 2 tracker provides during the time interval a signal indicating fitness data (e.g., 8 k steps per day) to member 2 (the challenged member) mobile communication device.

In block 424a member 1 (the challenging member) mobile communication device during a challenge time interval, in response to its internal timer periodically (e.g., daily) provides a signal containing challenge fitness data (e.g., 10 k steps) to the server.

In block 424b member 2 (the challenged member) mobile communication device during a challenge time interval, in response to its internal timer periodically (e.g., daily) provides a signal that includes challenge fitness data (e.g., 8 k steps) to the server.

In block 426, the server communicates via the communication network to the mobile communications device of the challenged user a leveled fitness metric to a challenged user. The level fitness metric of the challenging user is 1. The leveled fitness metric is determined for the challenged user based on the baseline fitness activity per periodic time period for the challenging user (e.g., 8 k steps average daily rate) divided by the baseline fitness activity per periodic time period for the challenged user (e.g., 6 k steps average daily rate) (e.g., resulting in a 1.33 leveling metric for the challenged user).

In block 436, the server communicates after a fitness activity from the one or more personal fitness devices to one or more mobile communication devices of the challenging user and the challenged user results of the tracking the fitness activity. Also, the server communicates with the one or more communications devices of the challenging user and the challenged user via a communications network to one or more servers, the fitness activity occurring during the duration of the challenge of the challenging user and the challenged user.

Also in block 436, in response to the timer (e.g., timer 207) indicating the end of the challenge, aggregating with one or more servers the results of the fitness activities of the challenge of the challenging user (e.g., 100 k steps over 10 days) and the challenged user (e.g., 800 k steps over 10 days) occurring during the duration of the challenge. The server also computes a challenged fitness metric of the challenging user by multiplying the fitness of the challenged user (e.g., 1) times the number of the challenger's physical activity (100 k) (100 k steps times by 1=100 k steps) (Block 438a). The server multiplies the fitness metric of the challenged user which was determined based on fitness activity of the challenged user during the duration of the challenge (e.g., 80 k steps) times the baseline fitness activity per periodic time period for the challenged user with respect to the challenging user (e.g., 1.33)(Block 438b). As the leveled fitness activity (e.g., 80 k×1.33=106 k steps) for member 2 is greater than the fitness activity (e.g., 100 k×1=100 k steps) of member 1, member 2 would win the challenge.

In block 440, the server determines and provides a signal to notify a winner to the mobile communication device of the challenging user and of the challenged user based on the challenged fitness metric as computed in block 436.

While the above detailed description has shown, described and identified several novel features of the invention as applied to a preferred embodiment, it will be understood that various omissions, substitutions and changes in the form and details of the described embodiments may be made by those skilled in the art without departing from the spirit of the invention. Accordingly, the scope of the invention should not be limited to the foregoing discussion but should be defined by the appended claims.

What is claimed is:

1. A method for metric leveling of personal fitness activity with one or more personal fitness devices communicating over a communications network to one or more mobile communications devices during a personal fitness wager event, the method:
tracking with the one or more personal fitness devices the personal fitness activity of multiple users;
communicating from the one or more personal fitness devices to the one or more mobile communication devices a signal indicating results of the tracking the personal fitness activity;
transmitting with a timer circuit coupled with the one or more mobile communication devices a signal indicating one or more end times of periodic time periods in which the fitness activity is to occur;
in response to the indication of the one or more end times by the timer circuit, communicating with the one or more communications devices via the communications network to one or more servers, at the end times of the periodic time periods aggregated a signal indicating results of the fitness activity over a periodic time period;
aggregating into a memory with one or more servers the results of the fitness activities occurring over a second predetermined time period;
computing with the one or more servers a baseline fitness activity per periodic time period for each of the multiple users;
receiving from at least one of the multiple users a signal indicating a fitness challenge on one of the mobile communication devices, the fitness challenge indicating fitness goal for completion of at least one fitness activity over a third predetermined time period;
communicating to the one or more servers and the mobile communication devices via the communication network a signal indicating a challenge by one of the mobile communications devices indicating a fitness challenge by a challenging member of one of the multiple users;
communicating a signal indicating a challenge acceptance to the one or more servers via the communication network by one of the mobile communication devices acceptance of the fitness challenge by a challenged user of one of the multiple users:
communicating by the server via the communication network to the mobile communications device of the challenged user a signal indicating a leveled fitness metric to a challenged user, the leveled fitness metric determined based on the baseline fitness activity per periodic time period for the challenging user and the baseline fitness activity per periodic time period for the challenged user;
storing in the server an indication of the duration of the challenge and setting with the server a timer to indicate the end of the challenge based on the stored duration;
in response to setting the timer, communicating by the server via the communication network to the mobile communications device of the challenging user and the challenged user a signal indicating of initiation of the challenge and the challenge duration;
tracking with one or more personal fitness devices the fitness activity of the challenging user and the challenged user;
communicating after the fitness activity from the one or more personal fitness devices to one or more mobile communication devices of the challenging user and the challenged user a signal indicating results of the tracking the fitness activity;
communicating, with the one or more communications devices of the challenging user and the challenged user via a communications network to one or more servers, a signal indicating fitness activity occurring during the challenge duration of the challenging user and the challenged user;
in response to the timer circuit indicating the end of the challenge, aggregating with one or more servers the results of the fitness activities of the challenge of the challenging user and the challenged user occurring during the challenge duration and computing a challenged fitness metric of the challenging user and of the challenged user based on fitness activity of the challenging user and the challenged user during the challenge duration and based on the baseline fitness activity per periodic time period for the challenging user and of the challenged user; and
providing a signal indicating a winner to the mobile communication device of the challenging user and of the challenged user based on the computed challenged fitness metric.

2. The method as recited in claim 1 wherein the timer circuit includes a crystal coupled with a counting circuit.

3. There method as recited in claim 1, wherein providing the signal indicating a winner to the mobile communication device of the challenging user and of the challenged user based on the challenged fitness metric includes providing a signal indicating whether the challenged fitness metric of the challenging user or the challenged fitness metric of the challenged user is greater.

4. An apparatus comprising:
a computer system having a timer circuit providing a signal indicating a predetermined baseline period of time; and
computer-readable media storing program code which when executed on the computer system performs the functions of:
determining from the signal the predetermined baseline period of time;
receiving a tracking signal from one or more mobile communications devices indicating fitness activity recorded on personal fitness devices of multiple users over the predetermined baseline period of time;
communicating a signal containing a challenge indication by one of the mobile communications devices indicating a fitness challenge to occur over a predetermined challenge period of time by a challenging user of one of the multiple users to a challenged user of one of the multiple users;
computing challenged fitness metrics of the challenging user and of the challenged user based on fitness activity of the challenging user and the challenged user occurring during the challenge period of time and during the baseline period of time; and
communicating to the one or more mobile communication devices a signal indicating one or more of the challenged fitness metrics upon completion of the challenge period of time to indicate a winner of the challenge.

6. The apparatus as recited in claim 5 wherein the timer circuit includes a crystal clock coupled with a counting circuit.

7. A system for leveling metrics between contestants in a fitness betting challenge comprising:
a plurality of personal fitness devices to be worn by a plurality of users, the fitness devices comprising means for determining with a timer circuit a predetermined baseline period of time, recording fitness activity of one of the plurality of users with one of a plurality of fitness devices during the predetermined baseline period of time and transmitting a signal indicating the recorded fitness activity;
one of a plurality of mobile communications devices having a receiver to receive from one of the plurality of fitness devices a signal indicating fitness activity recorded on the one of the plurality of personal fitness devices during the predetermined baseline period of time, and having a transmitter to transmit a signal with a challenge indication indicating a fitness challenge during a predetermined challenge period of time by a challenging user of the plurality of users to a challenged user of one of the multiple users;
a server computer comprising:
a receiver to receive a tracking signal from one of the plurality of mobile communications devices indicating fitness activity recorded on one of the personal fitness devices;
a transmitter to communicate the signal with the challenge indication by one of the mobile communications devices indicating a fitness challenge during the predetermined challenge period of time by a challenging user of one of the multiple users to a challenged user of one of the multiple users;
a processor to compute challenged fitness metrics of the challenging user and of the challenged user based on fitness activity of the challenging user and the challenged user occurring during the challenge period of time and during the baseline period of time; and
a transmitter to communicate to one of the mobile communication devices a signal indicating one or more of the challenged fitness metrics upon completion of the challenge period of time and an indication of a winner of the challenge.

8. The system as recited in claim 7, wherein the processor computes the challenged fitness metrics of the challenging user and of the challenged user based on a percentage of improvement of fitness activity of the challenging user and the challenged user during the challenge period of time from the baseline fitness activity for the challenging user and of the challenged user during the baseline period of time.

9. The system as recited in claim 7, wherein the mobile communications device includes means for indicating the winner of the challenge.

10. The system as recited in claim 7, wherein the personal fitness device includes a watch with a global positioning sensor used to track distance of the one of the multiple users during physical activity.

11. The system as recited in claim 7, wherein the personal fitness device includes at least one of a heart rate monitor, a pedometer, a GPS (global positing system) tracking device, a stopwatch, a blood level monitoring device, blood pressure monitor, body temperature monitor, caloric intake monitor, body fat percentage monitor, and a weight monitor.
